(19) United States
(12) Patent Application Publication
(10) Pub. No.: US 2022/0134030 A1
(43) Pub. Date: May 5, 2022

(54) PATHOGEN SENSING ADAPTORS FOR USE IN BREATHING CIRCUITS

(71) Applicant: COVIDIEN LP, Mansfield, MA (US)

(72) Inventors: Julio Jenaro, Galway (IE); Michael McGuinness, Galway (IE)

(21) Appl. No.: 17/511,083

(22) Filed: Oct. 26, 2021

Related U.S. Application Data
(60) Provisional application No. 63/108,677, filed on Nov. 2, 2020.

(51) Int. Cl. A61M 16/00; A61M 16/20

(52) U.S. Cl. CPC A61M 16/0003; A61M 16/208; A61M 2230/40; A61M 2205/584; A61M 2205/3306

(57) ABSTRACT

A pathogen detection system includes a pathogen sensing adaptor that detect pathogens present in the breathing circuit associated with a ventilated patient. A pathogen sensing adaptor may include a conduit, removable cartridges with testing strips, an optical sensor, and communication circuitry. Upon detecting a colorimetric change on the testing strip, the optical sensor generates a signal indicative of the presence and/or level of pathogens.

### CROSS REFERENCE TO RELATED APPLICATIONS

This application claims priority from and the benefit of U.S. Provisional Application Ser. No. 63/108,677, entitled “PATHOGEN SENSING ADAPTORS FOR USE IN BREATHING CIRCUITS,” filed Nov. 2, 2020, which is hereby incorporated by reference in its entirety for all purposes.

### BACKGROUND

The present disclosure relates generally to medical devices and, more particularly, to systems, devices, and related methods for detecting pathogens in respiratory circuits of ventilators associated with intubated patients.

This section is intended to introduce the reader to various aspects of art that may be related to the present disclosure, as described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air or other gases through a patient's trachea. Indeed, a medical provider may couple a ventilator to an exposed end of the tube and utilize the ventilator to mechanically control the type and amount of gases flowing into and out of the patient's airway. Intubated patients may be infected with contagious pathogens that are present in the lungs and in the patient's exhalation stream. In cases of infection, it may be helpful to identify pathogens present in the patient's inhalation and/or exhalation stream to determine suitable treatment options to cure the infection. However, testing for potential contagious pathogens in a breathing circuit may be costly, complex, and/or time-consuming. Further, the complexity and cost of the testing may result in reactive approaches to detecting pathogens such that testing is conducted after a patient has experienced symptoms from the infection.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, system for detecting pathogens in intubated patients is provide. The system may include a ventilator and a pathogen sensor adaptor that includes a conduit, a removable cartridge, an optical sensor, and communication circuitry. The ventilator may be connected to a breathing circuit that transfers ventilatory gases to and from a patient. The pathogen sensor adaptor may be in-line with the breathing circuit such that the conduit receives the ventilatory gases from the breathing circuit and passes the ventilatory gases to an adjacent portion of the breathing circuit such that ventilatory gases flow through the conduit. The removable cartridge includes a testing strip that is fluidically coupled to an interior space of the conduit. The testing strip also includes reagents that, when contacted with a pathogen in the interior space of the conduit, generate a colorimetric change on the testing strip. An optical sensor that is aligned with a test pad of the testing strip may generate a signal indicative of a presence or a level of the colorimetric change. In turn, the communication circuitry may communicate the signal to a controller of the ventilator.

In a further embodiment, a method of pathogen detection in a breathing circuit is provided. According to the method, instructions that activate a removable cartridge with a testing strip may be generated. In response to the instructions, the removable cartridge is actuated to align at least a portion of the testing strip with an optical reader such that the aligned portion of the testing strip is fluidically connected to an interior of the breathing circuit. Ventilatory gases of the breathing circuit flow past the testing strip. After the optical sensor detects a colorimetric change on the aligned portion of the testing strip and sends a signal indicative of the colorimetric change to pathogen sensing adaptor. The pathogen sensing adaptor may generate a notification related to a presence of level of a pathogen based on the signal.

In an additional embodiment, a pathogen sensing apparatus may include a conduit, a plurality of removable cartridges, and optical sensor, and communication circuitry. The conduit may include a first connector that couples to a first portion of a breathing circuit and a second connector that couples to a second portion of the breathing circuit such that ventilatory gases from a ventilator flow from the first portion of the breathing circuit to the second portion of the breathing circuit through the conduit. The plurality of removable cartridges may be inserted into respective slots of a plurality of slots formed in a wall of the conduit. Each removable cartridge of the plurality may include a testing strip fluidically connected to an interior space of the conduit. The testing strip may include reagents that, when contacted with a pathogen in the interior space of the conduit, generate a colorimetric change on the testing strip. The optical sensor that is fixedly coupled to the conduit at each of the plurality of slots may generate a signal indicative of a presence or level of the colorimetric change. In turn, communication circuitry may communicates the signal of each optical sensor to a controller of a ventilator.

Features in one aspect or embodiment may be applied as features in any other aspect or embodiment, in any appropriate combination. For example, any one of a system, monitor, ventilator, controller (e.g., processor-based controller), pathogen sensing apparatus, or method features may be applied as any one or more other of system, monitor, ventilator, controller, pathogen sensing apparatus, or method features.

### BRIEF DESCRIPTION OF THE DRAWINGS

Advantages of the disclosed techniques may become apparent upon reading the following detailed description and upon reference to the drawings in which:

FIG. 1 is a schematic illustration of a ventilated patient and a ventilation system, in accordance with certain embodiments of the disclosure;

FIG. 2 is a block diagram of an implementation of the ventilation system of FIG. 1 with a pathogen sensing adaptor, in accordance with certain embodiments of the disclosure;

FIG. 3 is a schematic illustration of a breathing circuit and the pathogen sensing adaptor of FIG. 2, in accordance with certain embodiments of the disclosure;

FIG. 4 is a schematic illustration of the pathogen sensing adaptor of FIG. 2, in accordance with certain embodiments of the disclosure;

FIG. 5 is a schematic illustration of the pathogen sensing adaptor of FIG. 2 with multiple removable cartridges, in accordance with certain embodiments of the present disclosure;

FIG. 6 is a schematic illustration of an embodiment of a pathogen sensing adaptor and corresponding continuous testing strip, in accordance with certain embodiments of the present disclosure;

FIG. 7 is a schematic illustration of a testing strip of FIG. 6 that includes nonabsorptive spacers, in accordance with certain embodiments of the present disclosure;

FIG. 8 is a flow diagram of a method to detect pathogens in intubated patients using the pathogen sensing adaptor of FIG. 2, in accordance with certain embodiments of the present disclosure;

FIG. 9 is a block diagram of the pathogen sensing adaptor of FIG. 2 communicatively coupled to a ventilator, in accordance with certain embodiments of the present disclosure; and

FIG. 10 is a schematic illustration of an example testing strip of a removable cartridge of the pathogen sensing adaptor of FIG. 2, in accordance with certain embodiments of the present disclosure.

### DETAILED DESCRIPTION OF SPECIFIC EMBODIMENTS

Pathogen sensing systems and methods are provided herein to detect pathogens in respiratory or breathing circuits of ventilators in or near real-time. An intubated patient or a patient assisted with a ventilator may develop lung or upper airway infections. In cases of infection, it may be useful to identify pathogens present in the patient's inhalation and/or exhalation stream to determine suitable treatment options to address the infection. Further, certain identified pathogens may be associated with enhanced isolation requirements for the infected patient to protect healthcare workers. However, acquiring samples of patient secretions (e.g., mucus, saliva) and testing the samples in vitro or remotely for pathogens may be complex, time-consuming, costly, and inefficient. As such, in situ detection of pathogens using a pathogen sensing adaptor may improve efficiency in detecting pathogens in intubated patients proactively and thereby improve determining suitable treatment options to address the infection. Rather than conducting pathogen detection tests after a patient has experienced symptoms from an infection, the in situ detection techniques of the pathogen sensing adaptor enable periodic or continuous testing. Such real-time and automated testing may enable patients to be treated before even before the patients experience symptoms from the infection.

In certain embodiments, the pathogen sensing adaptor is an in-line adaptor that includes a conduit that may be placed in-line (e.g., in series) with the tubing of a breathing circuit. The conduit includes an inlet that receives ventilatory gases from the breathing circuit and an outlet that passes the ventilatory gases to an adjacent portion of the breathing circuit such that ventilatory gases flow through the conduit. Thus, the pathogen sensing adaptor may be incorporated into an existing or conventional breathing circuit to add real-time pathogen sensing functionality. In embodiments, the disclosed in-line pathogen sensing adaptor provides benefits relative to sidestream-type sensing devices in which a portion of the gas flow within the breathing circuit is siphoned away from the breathing circuit via a coupled lumen and provided to a remote sensor positioned outside of the breathing circuit. While sidestream sensing may provide data representative of gases flowing in the breathing circuit, sidestream sensing may be less likely to capture pathogens associated with respiratory droplets in the breathing. These respiratory droplets are heavier than gases and have a tendency to be retained on surfaces, even in an environment of directional gas flow in the breathing circuit. Thus, pathogens in respiratory droplets may be underrepresented in sidestream collections, which may lead to inaccurate sensing in a sidestream arrangement.

In some embodiments, the pathogen sensing adaptor may also include a removable cartridge that supports a testing strip that, in operation, is positioned to be within the breathing circuit. In an embodiment, the pathogen sensing adaptor and/or the removable cartridge are disposable and replaceable. In an embodiment, the pathogen sensing adaptor includes cartridges that are integral with (e.g., are non-removable from) the pathogen sensing adaptor, such that the entire unit is replaced to provide new cartridges and/or update the pathogen sensing functionality. Thus, the conduit and integrated cartridge may be selected by the user based on the sensing characteristics of the cartridge.

Further, in certain embodiments, the conduit and cartridges may be provided together as a kit. In embodiments in which the cartridges are removable, the removable cartridges may be provided as separate components of the kit that may be selected according to the needs of the user. Thus, in an embodiment, the conduit is a universal receiver for different types of cartridges that may sense a variety of different types of pathogens. The conduit may be reusable (e.g., with the same patient or between patients after sterilization) while the cartridges are replaced.

Further, the testing strip of the removable or non-removable cartridges may include a sample pad or region that is exposed to the respiratory droplets of the gas flow within the breathing circuit. The pathogen sensing adaptor may also include an optical sensor, communication circuitry, and the like to detect potential pathogens within the conduit and alert medical personnel regarding detected pathogens. A portion of the removable cartridge may be inserted into an interior space of the conduit, while the remaining portion of removable cartridge may protrude outwards or be accessible from an exterior side of conduit. When activated, the removable cartridge may include a testing strip that is fluidically coupled to an interior space of the conduit. User input (e.g., pushing a button associated with the removable cartridge) or automatic actuation of the removable cartridge may cause a portion of the testing strip and corresponding portion of the removable cartridge to be inserted into the conduit. In response to activating the removable cartridge, the testing strip is aligned with an optical sensor that is stationary or fixedly attached to the conduit of the pathogen sensing adaptor.

The testing strip of the removable cartridge includes reagents that generate a colorimetric result in response to contact with a pathogen of interest that is in an interior space of the conduit. In some embodiments, the testing strip may include reagents for detecting a single type of pathogen of interest. In other embodiments, the testing strip may include reagents for detecting multiple types of pathogens. For example, different portions of the testing strip may be designated to identify a respective different type of pathogen. The reagents may be reagents used to perform polymerase chain reaction (PCR), loop-mediated isothermal amplification (LAMP), antibody tests, and/or other rapid diagnostic tests to produce a colorimetric result in response to detecting pathogens. For example, with respect to detecting pathogens using PCR techniques, the reagents may include primers with specificity for a nucleic acid of a particular type of pathogen of interest. Further, the reagents may include antibodies specific for a marker of the particular type of pathogen.

The testing strip of the pathogen sensing adaptor generates a colorimetric result in response to detecting a pathogen that is detected by an aligned optical sensor. The optical sensor, which is fixedly attached to the conduit and aligned with the testing strip, detects light (e.g., fluorescent light, visible light) from the colorimetric result on the testing strip. Based on the light detected, the optical sensor generates a signal indicative of a presence or a level of colorimetric change on the testing strip. The presence or the level of colorimetric change in turn is indicative of the presence, type, and/or number of pathogens detected in the conduit. The pathogen sensing adaptor, via the communication circuitry (e.g., Wi-Fi, Bluetooth, USB), communicates the sensor signal, or sends a notification related to a presence or number of pathogens based on the signal, in near or real time to a controller of coupled device, such a ventilator. In an embodiment, the signal may be indicated directly on the pathogen sensor adaptor device, e.g., via an on-board display or visible indicator. In response to receiving the signal or the notification, treatment options can be initiated that address infections caused by the detected pathogens. In some embodiments, in response to receiving the signal, the controller of the ventilator may dynamically change ventilator parameters to facilitate effective breathing in the presence of an infection.

Such in situ techniques and systems (e.g., including the pathogen sensing adaptor) to detect pathogens related to infections (e.g., severe acute respiratory syndrome coronavirus 2, pneumonia, tuberculosis, bacteria associated with ventilator associated pneumonia such as pseudomonas aeruginosa, escherichia coli, klebsiella pneumoniae, and acinetobacter species) in real-time while a patient is intubated may improve efficacy of treatment options and accelerate response time to treat infections. That is, the pathogen sensing adaptor may provide results indicative of the presence and/or level of pathogens in an intubated patient without acquiring a sample and physically removing the collected sample for remote testing. The pathogen sensing adaptor is capable of performing colorimetric tests (e.g., PCR, LAMP, antibody tests) that detect presence and/or level of pathogens while the removable cartridge is in-line with a breathing circuit of a ventilator. Dynamic detection of pathogens and respective infections while a patient is intubated may help medical personnel determine treatment options efficiently, and thereby increase chance of recovery while reducing hospitalization time for a patient.

Further an operator can service the pathogen sensing adaptor without disrupting the patient breathing circuit. For example, the testing strips and/or the removable cartridges of the pathogen sensing adaptors may be disposable. As reagents are depleted in a testing strip or strip, the testing strip may be replaced with a new testing strip without modification to the breathing circuit or interrupting breathing of an intubated patient. Because the testing strip protrudes minimally into the interior of the breathing circuit, the pathogen sensing adaptor has little effect on airway resistance of the breathing circuit and does not increase the work of breathing. Another benefit of the pathogen sensing adaptor is that more costly components of the adaptor, such as the optical sensor and communication circuitry, may be reusable. That is, new removable cartridges can be inserted into the pathogen sensing adaptor to be used with the optical sensor and communication circuitry already in place. Using disposable and replaceable components, the pathogen sensing adaptor detects pathogens and alerts medical personnel in real time without modifying the breathing circuit or interrupting breathing of intubated patients.

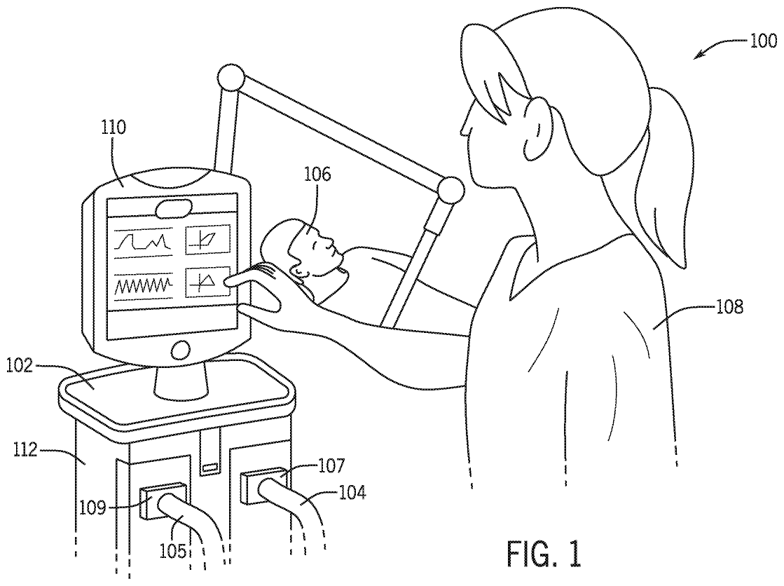

FIG. 1 is a ventilation system 100 that includes a ventilator 102 and a filter sterilization system as disclosed herein. As shown, the ventilation system 100 may include a ventilator 102. The ventilator 102 is used in conjunction with a ventilated patient 106 and by a clinician 108, who interacts with a display 110 of the ventilator 102 The ventilator 102 may engage one or more data collection sensors (not shown) to monitor various parameters that may be measured or calculated based on the closed system between the ventilator 102 and the patient 106. For example, the data collection sensors may collect one or more of gas flow, pressure, volume, or any other data or parameter that may be measured, calculated, or derived based on ventilation of the patient 106, measured at either or both the inhalation port 107 and exhalation port 109 of the ventilator. In an example, the ventilator 102 includes pressure and flow sensors at the inhalation port 107 that measure pressure and flow of the inhalation gases flowing into the inhalation limb 104 of a breathing circuit to the patient 106, and pressure and flow sensors at the exhalation port 109 that measure pressure and flow of the exhalation gases returning through the exhalation limb 105 of the breathing circuit to the ventilator from the patient 106. The ventilator 102 may also receive pressure and flow measurements from sensors along the breathing circuit, but these are optional. This measured, collected, or calculated data may be used by the clinician 108 or ventilator 102 when determining potential adjustments or changes to settings of the ventilator 102 in order to optimize patient-ventilator interaction. The breathing circuit is connected to a non-invasive interface (such as nasal prongs or a nasal, facial, or mouth mask) or an invasive interface (such as an endotracheal tube). The filter sterilization system as provided herein may be coupled to the ventilator 102 and in-line with the exhalation limb 105 at a point before (upstream of) the exhalation port 109, may be coupled after (downstream of) the exhalation port 109, may be integrated on or within a housing 112 of the ventilator 102, and/or may be provided as a modular component that couples to the ventilator 102.

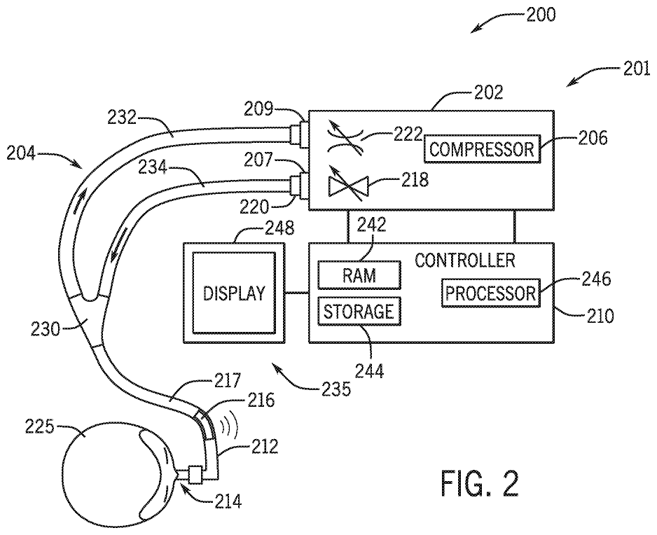

FIG. 2 is a block diagram of a ventilation system 201 that illustrates a ventilator 200 connected to a dual-limb breathing circuit 204 connected to an endotracheal tube 214, which is connected to a human patient 225. The breathing circuit 204 extends from the inhalation port 207 of the ventilator to the endotracheal tube 214, and from there back to the exhalation port 209 of the ventilator 200. The ventilator 200 controls the flow of gases into and out of the patient circuit by controlling (adjusting, opening, or closing) an inhalation flow valve 218 and an exhalation valve 222. Additionally, a humidifier 220 may be placed along the breathing circuit 204 to humidify the inhalation gases to enhance comfort for the patient 225. Pressure and flow sensors are located at the inhalation and exhalation ports 207, 209 to measure parameters of the inhalation and exhalation flows.

The ventilator 200 includes a pneumatic system 202 (also referred to as a pressure generating system 202) for circulating breathing gases to and from patient 225 via the breathing circuit 204 and the endotracheal tube 214. The breathing circuit 204 is a two-limb flexible tube for carrying gases to and from the patient 225. A fitting, typically referred to as a “wye-fitting” 230, connects an inhalation limb 234 and an exhalation limb 232 of the circuit, and couples the circuit to the endotracheal tube 214.

In some embodiments, a pathogen sensing adaptor 216 may include a conduit that is in-line or in-series with the breathing circuit 204. Any number of pathogen sensing adaptors 216 may be in-line with the breathing circuit 204. The pathogen sensing adaptors 216 may be in-line with or disposed in the inhalation limb 234, exhalation limb 232, and/or a tubing of the breathing circuit 204 located after the “wye-fitting” 230. For example, if the pathogen sensing adaptors 216 is in-line with the tubing of the breathing circuit 204 located after the “wye-fitting” 230, the conduit may connect a first portion 212 and a second portion 217 of the tubing of the breathing circuit 204. That is, the conduit of the pathogen sensing adaptor 216 includes a first connector that couples to the first portion 212 of the breathing circuit 204 and a second connector that couples to the second portion 217 of the breathing circuit 204 such that ventilatory gases from the ventilator 200 flow from the first portion 212 of the breathing circuit 204 to the second portion 217 of the breathing circuit 204 through the conduit.

The pathogen sensing adaptor 216 may include or be configured to operate with any suitable number of removable cartridges. Each of the removable cartridges may include a disposable testing strip or strip. A portion of the removable cartridge may be inserted into an interior space of the conduit while the remaining portion of the removable cartridge may protrude outwards from the conduit. The conduit of the pathogen sensing adaptor 216 may have any suitable inner or interior diameter (e.g., 10 mm, 15 mm). To prevent increasing the work of breathing for the patient 225, a protrusion or insertion of the removable cartridge of the pathogen sensing adaptor 216 into the interior space of the conduit may be less than 10% of an inner diameter of the conduit or less than 3 mm.

The inhalation limb 234 is connected to the inhalation port 207 and to the inhalation flow valve 218, and the exhalation limb 232 is connected to the exhalation port 209 and the exhalation flow valve 222. A compressor 206 or other source(s) of pressurized gases (e.g., tanks or hoses that supply compressed air, oxygen, and/or helium) provides a gas source for ventilatory support via inhalation limb 234. The pneumatic system 202 may include a variety of other components, including mixing modules, valves, sensors, tubing, accumulators, filters, etc. A controller 210 is operatively coupled with pneumatic system 202, signal measurement and acquisition systems, and an operator interface 235 that may enable an operator to interact with the ventilator 200 (e.g., change ventilator settings, select operational modes, view monitored parameters, etc.). The controller 210 may include hardware memory 242, one or more processors 246, storage 244, and/or other components of the type commonly found in command and control computing devices. In the depicted example, operator interface 235 includes a display 248 that may be touch-sensitive and/or voice-activated, enabling the display 248 to serve both as an input and output device.

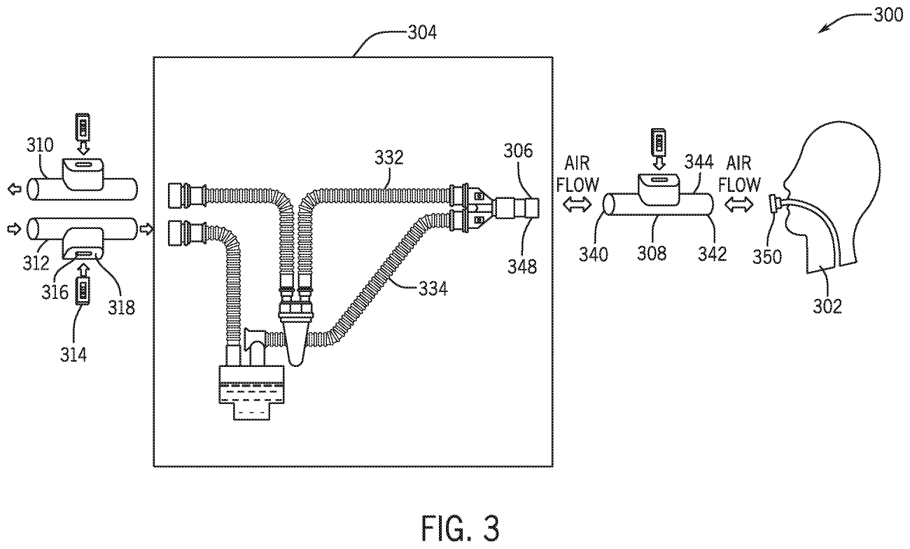

FIG. 3 is a schematic illustration of a pathogen detection system 300 that includes a ventilator associated with an intubated patient 302, a breathing circuit 304, and pathogen sensing adaptors 308, 310, and 312. As mentioned above, the breathing circuit 304 is coupled to the ventilator for carrying gases to and from the intubated patient 302. A fitting, typically referred to as a “wye-fitting” 306, connects an inhalation limb and an exhalation limb of the breathing circuit 304.

As illustrated, the pathogen sensing adaptors 308, 310, and 312 include respective removable cartridges 314 that are in uninserted configurations. The uninserted configurations of the removable cartridges indicate that respective portions of the removable cartridges and corresponding testing strips have not been inserted into respective interior spaces of conduits of the pathogen sensing adaptors 308, 310, and 312. For example, a slot or passageway 316 is formed in a housing 318 of the pathogen sensing adaptor that receives a removable cartridge 314.

As shown in FIG. 3, the pathogen sensing adaptor 308 is in-line with the tubing of the breathing circuit 304 located after the “wye-fitting” 306. The pathogen sensing adaptor 310 is in-line with the inhalation limb 334 of the breathing circuit 304. Further, the pathogen sensing adaptor 312 is in-line with the exhalation limb 332 of the breathing circuit 304. As mentioned above, any number of the pathogen sensing adaptors 308, 310, and 312 may be a part of the breathing circuit 304.

The pathogen sensing adaptor may include connectors 340, 342 at either end of a conduit 344 that are sized and shaped to connect to standard connectors, e.g., 15mm connectors, of the breathing circuit 304. For example, a first connector 340 connects to a connector 348 positioned distal to the wye fitting 306 and a second connector connects to an endotracheal tube connector 350. By providing standard connectors, the pathogen sensing adaptor may be positioned at any location in the breathing circuit 304, and in embodiments, multiple pathogen sensing adaptors may be directly coupled to each other as part of the breathing circuit 304. In an embodiment, the pathogen sensing adaptor may be coupled in different orientations without affecting function. For example, the connector 340 or the connector 342 may be positioned at a proximal or distal end of the pathogen sensing adaptor when coupled to the breathing circuit. The pathogen sensing adaptor may also be connected in a variety of rotational positions. Accordingly, the coupling to the breathing circuit 304 is user-friendly.

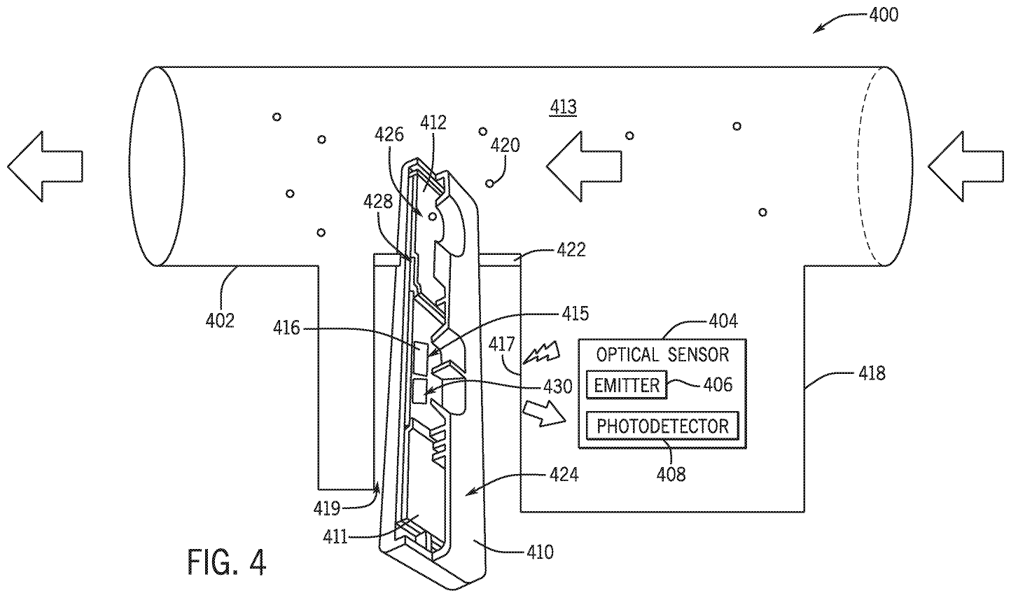

With the preceding in mind, FIG. 4 is a schematic illustration of a pathogen sensing adaptor 400 with a removable cartridge 410 in an inserted configuration that aligns a portion 415 of the testing strip 412 that generates a colorimetric change 416 with an optical sensor 404. In the inserted configuration of the removable cartridge 410, a portion of the removable cartridge 410 and corresponding testing strip 412 protrudes into an interior space 413 of a conduit 402 of the pathogen sensing adaptor 400. As illustrated, the conduit 402 connects a first portion (of a breathing circuit to a second portion of the breathing circuit such that ventilatory gases from a ventilator flow through from the first portion to the second portion through the conduit 402. In some embodiments, the conduit 402 may be transparent and may be composed of any suitable type of transparent material (e.g., polyvinyl chloride (PVC), polyethylene, thermoplastic elastomers (TPE), nylon, silicone). A cartridge receiver 418 that forms a slot or passageway 419 sized and shaped to receive the removable cartridge 410 is coupled to the conduit 402. The optical sensor 404 may be fixedly attached to a side or a wall of the conduit 402 and/or the cartridge receiver 418.

The optical sensor 404 detects light (e.g., fluorescent light, visible light) from a colorimetric result 416 on the testing strip 412. Thus, the optical sensor 404 and any transparent wall or material 417 of the pathogen sensing adaptor to which the optical sensor 404 is coupled permits light to be transmitted between the optical sensor 404 and the colorimetric result 416. Further, the transparent wall or material 417 prevents contact between the testing strip 412 and the optical sensor 404, thereby preventing any reagents, respiratory droplets 420, or other particulates from the testing strip 412 from contaminating the optical sensor 404. The optical sensor 404 includes an emitter 406 and a photodetector 408. The emitter 406 may be any suitable type of light source that emits electromagnetic radiation, e.g., ultraviolet (UV) light, fluorescent light, and/or visible light onto the colorimetric result 416 of the testing strip 412. Non-limiting examples of the emitter 406 may include light-emitting diode (LED). The emitted light from the colorimetric result 416 is reflected back to the optical sensor 404. The optical sensor 404 detects the reflected light from the colorimetric result 416 via the photodetector 408. The photodetector 408 may be any suitable type of light detector that detects electromagnetic radiation reflected back and/or generated by the colorimetric result 416. For example, the colorimetric change 416 may be the result of a chemical reaction that generates a fluorescent end product that is excited by the emitter 406 and that fluoresces at a wavelength detected by the photodetector 408. Non-limiting examples of the photodetector 408 may include phototransistors, photocells, light dependent resistors (LDRs), and image sensors.

When a pathogen is present in the breathing circuit or the conduit 402, and the pathogen contacts reagents of the testing strip 412, the testing strip 412 undergoes a chemical reaction to generate the colorimetric result 416. The optical sensor 404 may generate a signal indicative of this presence or level of the colorimetric change. The testing strip 412 may include any number or type of reagents. The testing strip 412 may be supported by or encased within the removable cartridge 410 that is coupled to the conduit 402. The removable cartridge 410 may be any suitable size and shape and may be composed of any suitable material. In some embodiments, a single removable cartridge 410 as illustrated provides housing for a single testing strip 412 that may detect multiple types of pathogens. Each different portion of the single testing strip 412 may be designated to detect a respective pathogen.

When the removable cartridge 410 is activated, a portion of the removable cartridge 410 may be inserted into the conduit 402 such that the testing strip 412 of the removable cartridge 410 is fluidically coupled to the interior space 413 of the conduit 402 and is able to directly contact respiratory droplets 420 associated with pathogens present in the respiratory gases. In some embodiments, user input (e.g., pushing a button associated with the removable cartridge 410) may activate the removable cartridge 410 and cause the removable cartridge 410 to be inserted into a slot or passageway of the conduit 402. In other embodiments, automatic actuation of the removable 410 in response to receiving or sensing pressure from patient secretions (e.g., mucus, saliva) may cause the removable cartridge 410 to be inserted into a slot or passageway of the conduit 402.

When the removable cartridge 410 is activated and thereby received in the slot or passageway 419 of the conduit 402, a one-way valve 422 coupled to the conduit 402 may open and enable the testing strip 412 of the removable cartridge 410 to be fluidically coupled to an interior space of the conduit 402 and aligned with the optical sensor 404. The one-way valve 422 may be a flap valve or may be formed of a self-sealing material that seals around the removable cartridge 410 so that the interior space 413 remains sealed against the ambient environment to maintain pressure within the breathing circuit. When the removable cartridge 410 is removed for disposal, the one-way valve 422 closes the passageway 419.

In some embodiments, the removable cartridge 410 includes a housing 424 that supports the testing strip 412, which may be implemented as an elongated structure in which separate areas corresponding to a sample pad or a contact region 426, an absorbent pad 411 that absorbs excess respiratory droplets 420, reagent pad 428 that includes reagent materials, a results portion 415 that generates the colorimetric change 416 and, in embodiments, a control line 430. Further, the testing strip 412 may be a lateral flow immunoassatry (LFIA) strip. The LFIA strip may collect the respiratory droplets 420 at the sample pad or a contact region 426. Further, the LFIA strip may be associated with pathogen detection technologies such as enzyme-linked immunosorbent assays (ELISA), enzyme multiplied immunoassay technique (EMIT), DNA reporters, real-time quantitative polymerase chain reaction (RT qPCR), real-time immunoquantitative PCR (iqPCR), and so forth. In other emdodiments, testing strip 412, may be implemented as an elongated structure without separate areas such that the colorimetric change 416 occurs at the contact region 426.

In certain embodiments, the housing 424 or the conduit 402 may include guides, such as stoppers or ridges that halt the movement of the testing strip 412 into the interior space to from sliding or being inserted into the interior space of the conduit 402 farther than a threshold depth. For example, the protrusion of the contact region 426 into the interior space 413 of the conduit 402 may be less than or equal to 3 mm, or less than 10% of an interior diameter of the conduit 402, to reduce changes to airway resistance in the breathing circuit. Further, the conduit 402 and/or the housing 424 may include alignment guides that enable the results portion 415 of the testing strip 412 to be in alignment with the optical sensor 404 and/or enable only the contact region 426 of the testing strip 412 to be inserted into the interior space of the conduit 402 while a rest of the testing strip 412 is retained beyond the interior space 413.

In an embodiment, the testing strip 412 of the removable cartridge 410 is encased in or protected by a breakable seal to prevent the testing strip 412 from being exposed to the ambient environment and thereby allow reagents in the testing strip 412 to remain fresh or unused. However, once the removable cartridge 410 is activated and received in the slot or passageway 419 of the conduit 402, the seal is broken to permit the pathogens in the interior space to come into contact with the testing strip 412. In one example, the cartridge receiver 418 includes an interior prong or protrusion that breaks the seal during insertion of the removable cartridge 410.

As provided herein, the testing strip 412 includes reagents that generate the colorimetric result 416 in response to the reagents contacting a pathogen in an interior space of the conduit 402. The testing strip 412 may include reagents for detecting one or more types of pathogens and/or one or more types of pathogen markers of a single pathogen. For example, each of the different portions of the testing strip 412 may be designated to identifying a respective type of pathogen. The reagents may be used to perform various in situ pathogen detection tests that generate the colorimetric result 416. Non-limiting examples of colorimetric pathogen detection techniques include polymerase chain reaction (PCR), loop-mediated isothermal amplification (LAMP), antibody tests (e.g., ELISA), gold and silver nanoparticles, and other rapid diagnostic tests that generate in colorimetric change the presence of pathogens. For example, with respect to detecting pathogen using PCR and LAMP techniques, reagents of the testing strip 412 may include primers with specificity for a nucleic acid of a particular type of pathogen. Further, for antibody testing, the reagents may include antibodies specific for a marker of the particular type of pathogen. Additionally, the reagents may include indicator dyes (e.g., picogreen hydroxynaphthol blue, propidium iodide) that change color after being in contact with pathogens, thereby helping generate the colorimetric result 416 that is indicative of the presence of pathogens after colorimetric pathogen detection techniques such as LAMP and PCR have been performed.

Different colorimetric pathogen detection techniques may have different reaction times, ranging from seconds to minutes. A permanently active optical sensor 404 may alter properties of the reagents or damage the reagents of the testing strip 412. For example, the heat produced from the optical sensor 404 may affect the reagents over time. As such, the optical sensor 404 may only be activated for relevant usage within the pathogen sensing adaptor 400. For example, the optical sensor 404 may be activated in response to the removable cartridge 410 being inserted into the slot or passageway 419 and the testing strip 412 being aligned with the optical sensor 404. The optical sensor 404 may obtain an optical reading (e.g., calibration result) in response to the testing strip 412 being initially aligned with the optical sensor 404. After obtaining the optical reading, the optical sensor 404 may be inactive until the completion of a colorimetric pathogen detection technique. For example, after the obtaining the optical reading, a controller of the optical sensor 404 may activate in a time-delayed manner associated with the detection technique. For example, if the testing strip is associated with LAMP, the optical sensor 404 may set a timer for at least 30 minutes after the removable cartridge 410 has been inserted into the slot or passageway of the conduit 402 and the optical reading has been obtained to reactivate the optical sensor 404 to detect the colorimetric result 416 generated from the performance of a colorimetric pathogen detection technique.

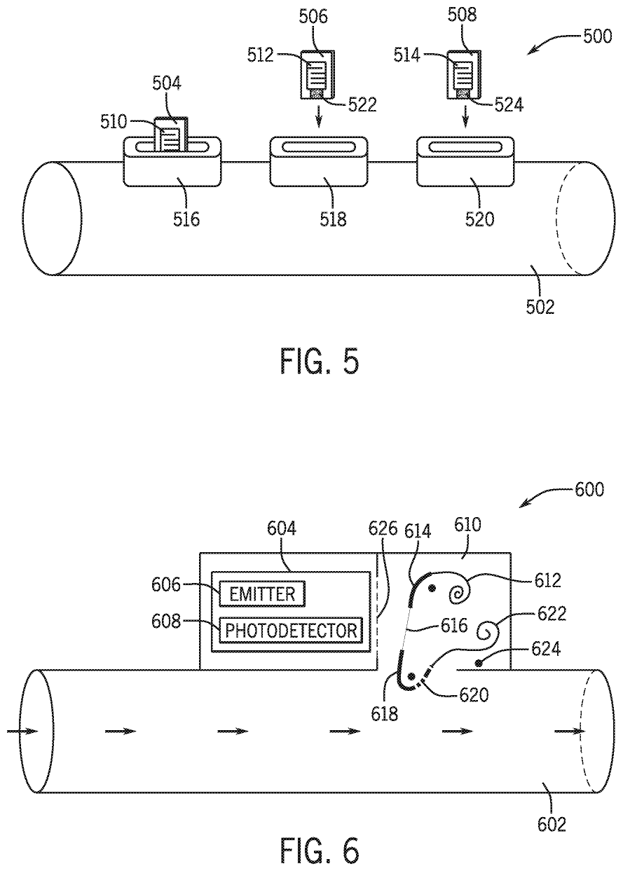

As mentioned above, a pathogen sensing adaptor may include any number of removable cartridges. FIG. 5 is a schematic illustration of a pathogen sensing adaptor 500 with a conduit 502 coupled to multiple removable cartridges 504, 506, and 508. The pathogen sensing adaptor 500 may include number and type of removable cartridges (e.g., removable cartridges 504, 506, and 508). Each removable cartridge and corresponding testing strip can be dedicated to detecting a particular type of pathogen. For example, removable cartridge 504, 506, and 508 provides housing for a respective testing strips 510, 512, and 514 that may be dedicated to detecting a particular type of pathogen.

In additional and/or alternative embodiments, each of the removable cartridges 504, 506, and 508 may be time-activated or time-separated. Testing for pathogen detection may be conducted by using a different removable cartridge at regular time intervals (e.g., every 30 minutes, every 1 hour, every 4 hours, every 8 hours) or at irregular time intervals. For example, with respect to irregular time intervals, 1 hour after a first removable cartridge is activated, a second removable cartridge may be activated. In turn, a third removable cartridge may be activated 3 hours after the first removable cartridge has been activated.

As shown in FIG. 5, the removable cartridge 504 has been activated and received by a slot or passageway 516 of the conduit 502. After activation, the removable cartridge 504 is in an inserted configuration. As such a portion of the removable cartridge 504 and respective testing strip 510 protrude into an interior space of the conduit 502. While the removable cartridge 504 is in an inserted configuration at a particular time period, the removable cartridges 506 and 508 are in uninserted configurations. As such, the removable cartridges 506 and 508 have not been activated nor been received by respective slots or passageways 518 and 520. As such, portions of the removable cartridges 506 and 508 and respective testing strips 512 and 514 have not been inserted into an interior space of the conduit 502. In the illustrated embodiment, the removable cartridges 506 and 508 are fully uninserted in the conduit 502. However, an inactive cartridge may be only partially inserted such that the testing strips are not within the interior space of the conduit 502. When the removable cartridges 506 and 508 are fully inserted into the conduit 502 (not shown), respective testing strips 512 and 514 are within the interior space of the conduit 502 such that respective sample pads 522 and 524 may collect respiratory droplets within the interior space of the conduit 502 that is in-line with a ventilatory gas path of a breathing circuit. In some embodiments, when the testing strips 512 and 514 are within the conduit 502, respective sample pads 522 and 524 may be within the interior space of the conduit 502, but other portions (e.g., reagent pad, results portion) of the testing strips 512 and 514 may not be within the interior space of the conduit 502.

FIG. 6 is a schematic illustration of an embodiment of a pathogen sensing adaptor 600 that includes a continuous testing strip 610 with multiple testing regions. In some embodiments, the continuous testing strip 610 may be a continuous LFIA strip. As such, each testing region on the continuous testing strip 610 may include separate areas such as an absorbent pad 614, a test pad 616 (e.g., control lines, test lines), a sample pad 618, isolating material 620, and the like. The continuous testing strip 610 may be a rolling or circular strip with any suitable thickness, length, and width. Based on user input to actuate the strip or automatic actuation of the strip, the strip may be rotated to expose a new testing region such that the test pad 616 of the new testing region is aligned with an optical sensor 604 of the pathogen sensing adaptor.

Rotating the continuous testing strip 610 involves exposing or unrolling a single testing region of the continuous testing strip 610 at a time such that the test pad 616 of the single testing region or the unrolled portion 612 is aligned with the optical sensor 604. As mentioned in detail above, the optical sensor 604 may include an emitter 606 and a photodetector 608. In some embodiments, a housing that supports the continuous testing strip 610 optical sensor 604 may include a transparent wall 626 (e.g., glass, PVC, TPE, nylon, silicone). The transparent wall 626 may serve as a physical divider between the optical sensor 604 and the test pad 616 for a particular testing region of the continuous testing strip 610. Thus, the optical sensor 604 and any transparent wall 626 of the pathogen sensing adaptor 600 to which the optical sensor 404 is coupled permits light to be transmitted between the optical sensor 404 and the test pad 616.

Further, unlike other portions of each testing region, the sample pad 618 of each testing region protrudes into an interior space of a conduit 602 to directly contact respiratory droplets 624 associated with pathogens present in the respiratory gases of a breathing circuit that is in-line with the conduit 602. After the respiratory droplets 624 are collected at the sample pad 618, the respiratory droplets 624 may be absorbed by the test pad 616, in which the respiratory droplets 624 react with reagents (e.g., antibodies) to generate a colorimetric change. From the test pad 616, the respiratory droplets 624 may propagate to other proceeding areas (e.g., the absorbent pad 614). The absorbent pad 614 serves to absorb excess respiratory droplets 624 that were not used at the test pad 616.

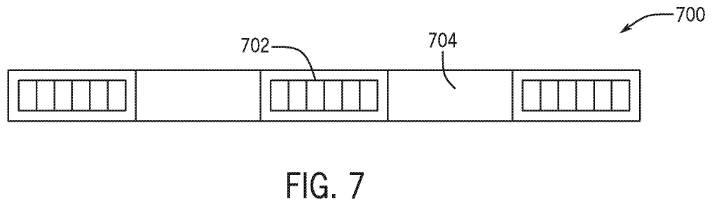

In some embodiments, the unrolled testing region 612 may be separated from the rolled testing region 622 via an isolating material or nonabsorptive spacers 620. Used testing regions with depleted reagents may be rolled up to form the rolled testing region 622. The nonabsorptive spacers will be further discussed in FIG. 7. In additional and/or alternative embodiments, the pathogen sensing adaptor 600 may include a non-capillary based continuous testing strip such that the colorimetric change occurs at a contact or collection site of the respiratory droplets 624. That is, each testing region of the non-capillary based continuous testing strip may not include separate areas such as the absorbent pad 614, test pad 616, and sample pad 620.

In some embodiments, each of the multiple testing regions may be dedicated to detecting a particular type of pathogen. A continuous testing strip 610 with multiple testing regions may decrease the number of removable cartridge replacements. That is, when reagents have been depleted, rather than inserting a new removable cartridge with a new testing strip, a new testing region of the circular, continuous strip of an existing removable cartridge may be used once reagents of a used testing region have been exhausted. Further, to detect a different type of pathogen, rather than replacing a removable cartridge that is dedicated to a particular pathogen, the circular, continuous strip with various testing regions that are dedicated to detecting different types of pathogens may be used. For example, the new testing region of the circular, continuous strip may be aligned with the optical sensor 604 in order to perform testing with respect to a different type of pathogen.

FIG. 7 is a schematic illustration of a continuous testing strip 700 with testing regions 702 and nonabsorptive spacers 704. While the testing strip 700 may be a continuous rolling or circular strip, an example portion of the continuous rolling or circular strip is depicted in FIG. 7. Each of testing regions 702 is separated by a nonabsorptive spacer 704 to separate the testing region 702 such that individual results may be distinguished by an optical sensor of the pathogen sensing adaptor. The nonabsorptive spacers 704 may be any suitable size and shape and may be composed of any suitable material such as plastic.

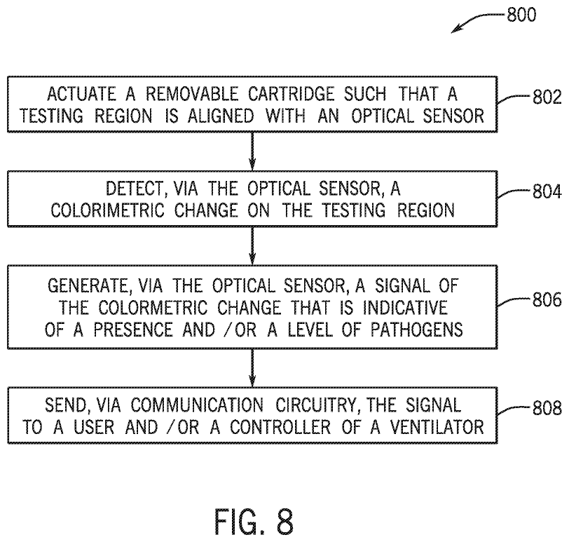

FIG. 8 is a flow diagram of a method 800 for detecting pathogens in intubated patients using pathogen sensing adaptors, in accordance with some embodiments. Certain steps of the method 800 are discussed in the context of elements referenced in FIG. 4. Further, certain steps or portions of the method 800 may be performed by separate devices and components, such as one or more components illustrated in FIG. 4.

The method 800 initiates with actuating the removable cartridge 410 of the pathogen sensing adaptor 400 such that the testing strip 412 is aligned with an optical sensor 404 at step 802. User input (e.g., pushing a button associated with the removable cartridge 410) or automatic actuation of the removable cartridge 410 in response to receiving patient secretions (e.g., mucus, saliva) may activate the removable cartridge 410 such that the removable cartridge 410 in an inserted configuration. According to the inserted configuration, a portion of the removable cartridge 410 and corresponding testing strip 412 protrude into an interior space of the conduit 402.

When pathogens are present in a breathing circuit associated with an intubated patient, and the pathogens come into contact with reagents of the testing strip 412, a colorimetric result is generated via colorimetric pathogen detection techniques (e.g., antibody tests, LAMP, PCR). As such, after the testing strip 412 is aligned with the optical sensor 404, the optical sensor 404 detects any colorimetric changes on the testing strip 412 at step 804. As described above, the optical sensor 404 detects fluorescent and/or visible light associated with the colorimetric change via emitter 406 and the photodetector 408.

At step 806, based on the colorimetric change, the optical sensor 404 generates a signal indicative of a presence and/or a level of pathogen. The presence or the level of colorimetric change may be indicative of the presence, type, and/or number of pathogens detected in the conduit.

Using communication circuitry (e.g., Wi-Fi module, Bluetooth module), the pathogen sensing apparatus 400 sends the signal to a user and/or a controller of a ventilator associated with an intubated patient at step 808. In response to receiving the signal, the user (e.g., medical personnel) may change ventilator parameters or initiate treatment options that address infections caused by the detected pathogens. In some embodiments, in response to receiving the signal, the controller of the ventilator may dynamically change ventilator parameters to facilitate effective breathing or further reduce the work of breathing by a patient in the presence of an infection.

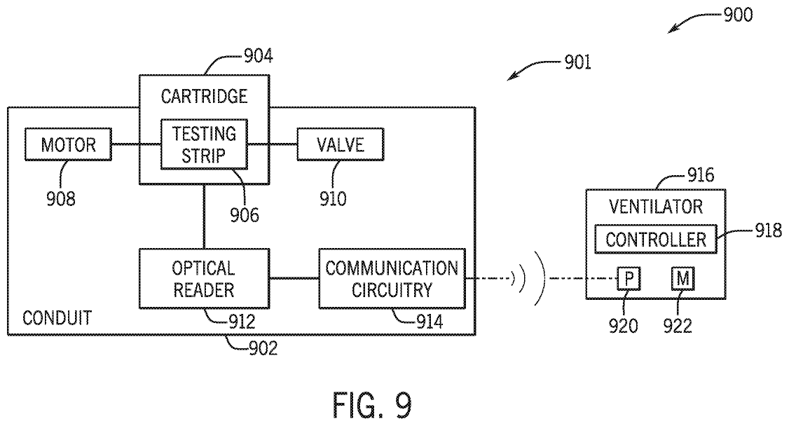

FIG. 9 is a block diagram of a pathogen detection system 900, in which a pathogen sensing adaptor 901 is communicatively coupled to a ventilator 916. As mentioned above, the pathogen sensing adaptor 901 includes a conduit 902, a removable cartridge 904 with a testing strip 906, and an optical reader or sensor 912. The pathogen sensing adaptor 901 may also include a motor or actuator 908 and a valve 910. In response to receiving instructions from a user or a controller 918 to activate the removable cartridge 904, motor or actuator 908 may actuate the removable cartridge 904 such that a portion of the removable cartridge 904 is inserted into an interior space of the conduit 902. When the removable cartridge 904 is activated and thereby received in the slot or passageway of the conduit 902, the one-way valve 910 coupled to the conduit 902 may open and enable the testing strip 906 of the removable cartridge 904 to be fluidically coupled to an interior space of the conduit 902 and aligned with the optical reader or sensor 912. Further, communication circuitry 914 of the pathogen sensing adaptor 901 includes a Wi-Fi module, Bluetooth module, universal serial bus (USB) to send a signal or notification related to a presence or number of pathogens based on the controller 918 of a ventilator or a user in response to the optical reader or sensor 912 detecting a colorimetric change.

In certain embodiments, the pathogen sensing adaptor 901 is communicatively coupled to a controller 918 of a ventilator 916. The controller 918 may execute hardware and/or software control algorithms to actuate components within the pathogen sensing adaptor 901 such as the removable cartridge 904. The controller 918 may include a programmable logic controller (PLC) or other suitable control device. According to some embodiments, the controller 918 may include an analog to digital (A/D) converter, one or more microprocessors or general or special purpose computers, a non-volatile memory, memory circuits, and/or an interface board. For example, the controller 918 may include memory circuitry for storing programs, control routines, and/or algorithms implemented for control of the various system components. The controller 918 also includes, or is associated with, input/output circuitry for receiving sensed signals from the optical reader or sensor 912. Memory circuitry may store set points, actual values, historic values, and so forth, for any or all such parameters. Any other suitable devices may be included in the pathogen detection system 900, such as additional transducers or switches that sense fluorescence and/or visible light from a colorimetric result, actuate components, and so forth.

The controller 918 also may include components for operator interaction with the systems, such as display panels and/or input/output devices for checking operating parameters, inputting control signals representative of set points and desired operating parameters, checking error logs and historical operations, and so forth. The controller 918 may receive data from the optical reader or sensor 912 and/or control the activation of the removable cartridge 904.

The controller 918 may include processor(s) 920 (e.g., a microprocessor(s)) that may execute software programs to control the pathogen detection system 900. Moreover, the processor 920 may include multiple microprocessors, one or more “general-purpose” microprocessors, one or more system-on-chip (SoC) devices, one or more special-purpose microprocessors, one or more application specific integrated circuits (ASICs), and/or one or more reduced instruction set computer (RISC) processors. The controller 918 may include a memory device 922 that may store executable instructions and/or information such as control software, look up tables, configuration data, etc.

The memory device 922 may include a tangible, non-transitory, machine-readable medium, such as a volatile memory (e.g., a random access memory (RAM)) and/or a nonvolatile memory (e.g., a read-only memory (ROM), flash memory, a hard drive, and/or any other suitable optical, magnetic, or solid-state storage medium). The memory device 922 may store a variety of information that may be used for various purposes. For example, the memory device 922 may store machine-readable and/or processor-executable instructions (e.g., firmware or software) for the processor 920 to execute.

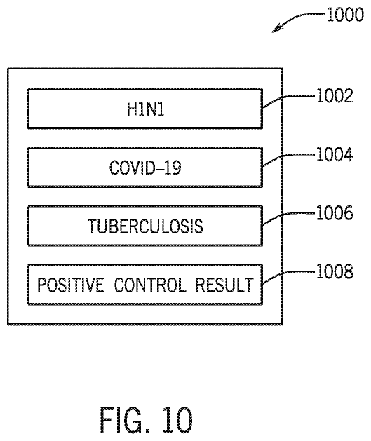

With the preceding in mind, FIG. 10 is a schematic illustration of an example testing strip 1000. As mentioned above, the testing strip 1000 may include various reagents for detecting different types of pathogens. Each of the different portions of the testing strip may be designated to identifying a respective type of pathogen. For example, the testing strip 1000 includes three different portions 1002, 1004, and 1006, that respectively correspond to pathogen testing for H1N1, COVID-19 (severe acute respiratory syndrome coronavirus 2), and tuberculosis. Additionally, the testing strip 1000 includes a portion 1008 for a positive control. The depicted embodiment is by way of example, and it should be understood that other arrangements and other types of pathogens are contemplated.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A system, comprising:
a ventilator coupled to a breathing circuit that transfers ventilatory gases to and from a patient; and
a pathogen sensor adaptor coupled to and in-line with the breathing circuit, wherein the pathogen sensor adaptor comprises:
a conduit comprising an inlet that receives the ventilatory gases from the breathing circuit and an outlet that passes the ventilatory gases to an adjacent portion of the breathing circuit such that ventilatory gases flow through the conduit;
a removable cartridge, wherein a portion of the removable cartridge comprising a testing strip is fluidically coupled to an interior space of the conduit, wherein the testing strip comprises reagents that, when contacted with a pathogen in the interior space of the conduit, generate a colorimetric change on the testing strip;
an optical sensor aligned with the testing strip and that generates a signal indicative of a presence or a level of the colorimetric change; and
communication circuitry that communicates the signal to a controller of the ventilator.

2. The system of claim 1, wherein the removable cartridge is removable from an exterior of the conduit.

3. The system of claim 1, wherein the conduit comprises a slot or passageway into which the removable cartridge is received.

4. The system of claim 3, comprising a one-way valve that opens to permit the testing strip to be fluidically coupled to the interior space of the conduit when the removable cartridge is received into the slot or the passageway and that closes to seal the slot or the passageway when the removable cartridge is removed.

5. The system of claim 1, wherein the reagents comprise one or more antibodies specific for a marker of the pathogen.

6. The system of claim 1, wherein the reagents comprise one or more primers with specificity for a nucleic acid of the pathogen.

7. The system of claim 1, wherein the optical sensor comprises an emitter and a photodetector that detects light from the emitter that has interacted with the testing strip.

8. The system of claim 7, wherein the optical sensor is fixedly attached to the conduit.

9. The system of claim 7, wherein the detected light is visible light or fluorescent light.

10. The system of claim 1, wherein the removable cartridge, when coupled to the conduit, protrudes into the interior space.

11. The system of claim 10, wherein the removable cartridge protrudes into the interior space an amount less than 10% of a diameter of the conduit.

12. The system of claim 10, wherein the conduit has an interior diameter of 15 mm, and wherein the removable cartridge protrudes into the interior space an amount less than 3 mm.

13. The system of claim 1, wherein the ventilator comprises the controller that receives the signal from the optical sensor and generates a notification based on the presence or the level of the colorimetric change.

14. A method of pathogen detection in a breathing circuit comprising:
generating instructions to activate a removable cartridge comprising a testing strip;
actuating the removable cartridge, in response to the instructions, to align at least a portion of the testing strip with an optical reader and such that the aligned portion of the testing strip is fluidically coupled to an interior of the breathing circuit, wherein ventilatory gases of the breathing circuit flow into the testing strip;
detecting a colorimetric change on the aligned portion of the testing strip via an optical sensor;
receiving a signal indicative of the colorimetric change; and
generating a notification related to a presence of level of a pathogen based on the signal.

15. The method of claim 14, comprising changing ventilation settings of a ventilator based on the notification.

16. The method of claim 14, wherein actuating the removable cartridge comprises rotating the testing strip to align an unused portion of the testing strip with the optical reader.

17. The method of claim 14, wherein the testing strip comprises a plurality of testing regions separated by nonabsorptive spacers, and wherein the aligning comprises moving a new testing region into alignment with the optical reader.

18. A pathogen sensing apparatus, comprising:
a conduit comprising a first connector that couples to a first portion of a breathing circuit and a second connector that couples to a second portion of the breathing circuit such that ventilatory gases from a ventilator flow from the first portion of the breathing circuit to the second portion of the breathing circuit through the conduit;
a plurality of cartridges within respective slots of a plurality of slots formed in a wall of the conduit, each cartridge of the plurality comprising:
a testing strip fluidically coupled to an interior space of the conduit, wherein the testing strip comprises reagents that, when contacted with a pathogen in the interior space of the conduit, generate a colorimetric change on the testing strip;
an optical sensor fixedly coupled to the conduit at each of the plurality of slots;
the optical sensor configured to generate a signal indicative of a presence or level of the colorimetric change; and
communication circuitry that communicates the signal of each optical sensor to a controller of a ventilator.

19. The pathogen sensing apparatus of claim 18, wherein the conduit comprises one or more alignment guides, one or more ridges, or both that guide alignment of at least a portion of the testing strip with the optical sensor.

20. The pathogen sensing apparatus of claim 18, wherein each of the plurality of cartridges is removable, and wherein each of the plurality of cartridges is activated or inserted into a respective slot of the plurality of slots at regular or irregular time intervals.
